(19) United States
(12) Patent Application Publication
(10) Pub. No.: US 2009/0030400 A1
(43) Pub. Date: Jan. 29, 2009

(54) SYSTEM AND METHOD FOR INTRACRANIAL ACCESS

(76) Inventors: Arani Bose, New York, NY (US); David Barry, Livermore, CA (US); Delilah Hui, American Canyon, CA (US); Ben Tompkins, Danville, CA (US)

Correspondence Address: STALLMAN & POLLOCK LLP, 353 SACRAMENTO STREET, SUITE 2200, SAN FRANCISCO, CA, 94111, US

(21) Appl. No.: 12/178,521

(22) Filed: Jul. 23, 2008

Related U.S. Application Data
(60) Provisional application No. 60/961,957, filed on Jul. 25, 2007.

(51) Int. Cl. A61M 25/088

(52) U.S. Cl. 604/510; 604/528

(57) ABSTRACT

A delivery catheter for accessing the intra-cranial vascular includes a rigid proximal section and a distal section having an outer diameter and flexibility suitable for advancement into the intra-cranial vasculature, such as the Petrous segment or the Cavernous segment of the internal carotid artery. The wall thickness and rigidity of the catheter decrease from the proximal section to the distal section, preferably in discrete segments each having reduced wall thickness and/or durometer relative to proximally adjacent sections. An intra-cranial access system includes the delivery catheter and a selection catheter insertable through the lumen of the delivery catheter. The selection catheter is shaped to facilitate selection of the target branch of the neurovasculature off the aortic arch and allows the delivery catheter to be advanced over the selection catheter into the selected branch.

### PRIORITY

This is application claims priority to U.S. Provisional Application No. 60/961,957, filed Jul. 25, 2007, which is incorporate herein by reference.

### TECHNICAL FIELD OF THE INVENTION

The present invention relates generally to the field of intravascular guide catheters. In particular, the invention relates to the field of guide catheters for accessing the neurovasculature.

### BACKGROUND

Guide catheters are used to introduce diagnostic or interventional devices into the peripheral, coronary, and neurovasculature.

Currently marketed neurovascular guide catheters are modeled after cardiovascular guide catheters. In fact, many neurovascular guide catheters are essentially cardiovascular guide catheters that have been modified with a more flexible, less traumatic distal segment.

This modification allows physicians to enter the neurovascular anatomy, but it is not optimized for the neurovasculature.

During use of a currently available neurovascular guide catheters, a standard sheath is placed in the femoral artery. The guide catheter is flushed with heparinized saline and a 0.038″ guidewire is introduced into the guide catheter. The guide catheter and 0.038″ guidewire are introduced through the sheath into the femoral artery. Under fluoroscopy, the system is advanced through the aorta into the aortic arch. While pulling back and torqueing the guide catheter or wire, the appropriate artery off the arch is selected. The 0.038″ guidewire and guide catheter are then advanced toward the selected neurovascular anatomy. For instance, if an intervention is to be performed in the anterior circulation, the guide catheter may be advanced to the proximal (downstream) end of the cervical internal carotid artery. Once the distal end of the guide catheter is so positioned, interventional devices (e.g. microcatheters, stents, PTA balloon catheters, and coils) are then introduced through the guide catheter, and passed out the distal end of the guide catheter for use in the desired treatment area.

A significant drawback to currently marketed neuro guide catheters is their tendency to “back out” during a procedure. In particular, resistive forces due to vessel tortuosity and diameter are encountered as a neurovascular device passed through a guide catheter is tracked through the intra-cranial anatomy. The resistance to forward movement generates an equal and opposite force that must be absorbed by the guide catheter, or else the guide catheter loses position and “backs out” from its position into the common carotid and then into the aorta. When this occurs, the physician must shift focus from accessing the treatment site with the neuro device to repositioning the guide catheter. This often requires that the angiographic field of view be adjusted away from the intra-cranial vasculature. In certain clinical settings, the physician may have to remove the interventional device, and reselect the neurovascular branch vessel off the aortic arch.

In many cases, the physician is burdened with this problem multiple times during the course of a single procedure.

One currently available solution to the issue of back-out is to replace the standard sheath with a long-sheath that extends from the femoral access site through the aorta into the common carotid artery. The guide catheter is then passed through the long sheath and positioned as discussed above. The added proximal support of the sheath can augment the forward axial transmission of force and reduce back-out. As an alternative solution, the surgeon may attempt to advance the guide catheter more distally into the carotid artery up to the skull base. Both of these options make the procedure more difficult and have potential safety issues due to vessel trauma.

Conventional neurovascular guide catheters are susceptible to the “backing out” phenomenon for the following reasons. First, as a result of their limited length and distal flexibility conventional guide catheters are parked in the proximal neuro-vasculature (in relatively straight vessels). Due to this position, a guide catheter is required to resist backward forces transmitted from a neuro device without the support of the surrounding anatomy. Second, conventional neurovascular guide catheters have a flexibility profile that is not optimized for intra-cranial access. The conventional flexibility profile includes a stiff, supportive proximal segment that abruptly transitions to a relatively flexible distal segment. This design is prone to back out because the backward force transmitted from a neuro device acts to load the flexible/stiff junction in such a way that the guide catheter springs back into the aorta. The disclosed system includes features that give access to the neuro-vasculature while providing greater support against catheter backout.

### BRIEF DESCRIPTION OF THE DRAWINGS

FIG. 1 is a side elevation view of an embodiment of an intracranial access system;

FIG. 2 is a cross-sectional side view of the catheter shown in FIG. 1;

FIG. 3 schematically illustrates positioning of the delivery catheter through the aortic arch to the Petrous segment of the internal carotid artery;

FIG. 4 is a schematic illustration of a human head comparing the positioning of the distal end of the delivery catheter as shown in FIG. 3 with the placement of the distal end of a delivery catheter using conventional procedures.

### DETAILED DESCRIPTION

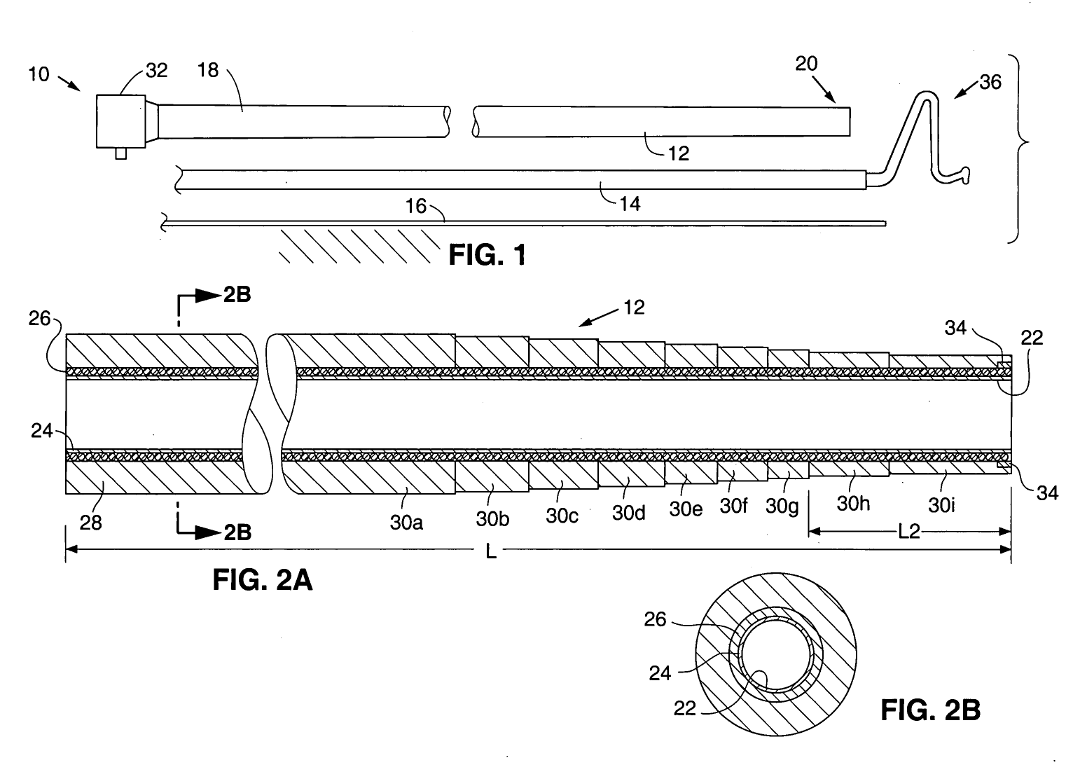

FIG. 1 illustrates an exemplary embodiment of an access system 10 for the intracranial vasculature. System 10 includes a delivery catheter 12, optional selection catheter 14, and guidewire 16. The system 10 may be packed as a kit containing all or a subset of these components, together with instructions for use instructing the user to use the system according to the method of use disclosed herein.

Delivery catheter 12 is constructed and proportioned to provide the proximal support of a long femoral access sheath from the femoral artery through the aortic arch, then mimics a traditional neurovascular guide catheter in the cervical region, and continues with the distal flexibility and kink resistance of a microcatheter into the intra-cranial vasculature with in the skull base. This flexibility profile gives physicians the ability to position the delivery catheter in tortuous regions of the neurovasculature (e.g. the Petrous or Cavernous segment of the internal carotid) where a typical guide catheter could not be utilized.

FIGS. 2A and 2B are cross-sectional views of a delivery catheter having one example of a flexibility profile that may be utilized.

In the illustrated embodiment, the catheter 12 includes a lumen 22 extending the length of the catheter. The lumen may have a tapered inner diameter or a uniform inner diameter (e.g. 0.053 in or greater). A lubricious inner liner 24, preferably formed of PTFE, lines the lumen 22, allowing for smooth passage of instruments through the lumen. A reinforcing layer 26 of coil or braid surrounds the liner 24 and provides the catheter with enhanced kink resistance, pushability, and torquability. In the FIG. 2A-2B embodiment the reinforcing layer 26 extends the full length of the catheter body, and is formed of stainless steel or platinum wire coiled over the PTFE liner at a uniform pitch. In other embodiments, the reinforcing layer may terminate proximal to the distal end of the catheter, and/or the pitch of the coil may vary along the length of the catheter.

Delivery catheter 12 includes an outer layer 28 formed of a number of polymer extrusions of varying durometer and wall thickness arranged such that the delivery catheter transitions smoothly from a 6F stiff proximal shaft segment 18 to a 5F microcatheter-like distal segment 20. The number of segments and the length, durometer, and outer diameter of the segments are selected such that properties of a segment are best suited for the regions of the neurovascular through which that segment of the catheter will pass during positioning and within which that segment of the catheter will be “parked” during use of an interventional device passed through the delivery catheter.

The FIG. 2A-2B embodiment includes nine segments 30a-30i. The most proximal three segments 30a, 30b, 30c are designed to be stiff to give sheath-like support within the aorta. A suitable material for the proximal segments 30a-30c is a stiff nylon material (e.g. approximately 80D-85D hardness), such as the Nylon 12 material sold under the trade name Vestamid L2101F. Although the segments 30a-30c are formed of the same material, they have progressively smaller wall thicknesses (e.g. from 0.009 in, 0.008 in and 0.006 in, respectively) to give the delivery catheter a smooth taper. Segments 30d-30i are selected to have progressively decreasing durometer and wall thickness, section 30d having the highest durometer and thickest wall and section 30i having the lowest durometer and thinnest wall. For example, the durometer of the outer layer 28 may range from 72D at segment 30d to 80A at the most distal segment 30i, with the wall thickness ranging from 0.005 in to 0.0027 in. Each of the intervening segments may progressively decrease in both wall thickness and durometer, or in one or the other. For example, in one embodiment each of the segments has a lower durometer than its proximally adjacent segment. In this example, each of segments 30d-30f has a thinner wall thickness than its proximately adjacent segment, segments 30f-30h have approximately equal wall thicknesses (e.g. 0.0035 in), and segment 30i has a thinner wall thickness than segments 30f-30h.

With the illustrated flexibility profile, the proximal segments 30a-30c provide proximal support similar to that of a long femoral access sheath from the femoral artery through the aortic arch, then mimics a traditional neurovascular guide catheter with segments 30d-30g in the cervical region, and at segments 30h and 30i continues with the distal flexibility and kink resistance of a microcatheter into the intra-cranial vasculature with in the skull base.

Suitable materials for the outer layer 28 include Pebax and polyurethane at the selected durometer. A hydrophilic coating such as Polyvinylpyrrolidone or Polyacrylamide may be formed over some or all of the segments, such as the more distal segments 30h-30i to minimize friction during advancement of the catheter through the vasculature.

In one embodiment, the delivery catheter may have an overall length L of 105-115 cm. One preferred delivery catheter has a length of approximately 105 mm, another has a preferred length of approximately 115 cm. Segments 30b-30g may each have lengths of approximately 2 cm, with proximal most segment 30a being significantly longer, and with the most flexible distal segments 30h, 30i having a combined length L2 of approximately 6-12 cm. In one preferred embodiment, L2 is approximately 6 cm. In another preferred embodiment L2 is approximately 12 cm. In these examples, the proximal segment 30a may have a length of approximately 80-100 cm (e.g. 81 cm, 87 cm, 91 cm, 97 cm). A conventional valve hub 32 (FIG. 1) and side arm may be provided at the proximal end of the delivery catheter. Radiopaque markers 34 (FIG. 2A) made of platinum iridium or other suitable materials may be included at or near the distal end to facilitate fluoroscopic visualization of the delivery catheter. In another embodiment, the outer layers of the distal segments 30h-30i could be doped with a radiopaque material (Barium Sulfate). In a further embodiment, the distal segment could use a platinum wire wound to a tight pitch to function as a marker.

The unibody construction and variable flexibility of the delivery catheter allows for a stable supportive path from the femoral artery up to the intra-cranial vasculature. The flexibility profile, and position in the neuroanatomy anchors the delivery catheter while optimizing its ability to absorb the resistive forces being transmitted back as complex neurovascular devices are navigated through distal intra-cranial tortuosity, This augmented support allows safer and more effective delivery of such devices into distal intra-cranial target vessels, thereby allowing the operator to focus on delivering devices to a treatment site that might otherwise be inaccessible.

Referring again to FIG. 1, the selection catheter 14 is proportioned for insertion through the lumen of the delivery catheter. The selection catheter 14 includes a distal tip section 36 shaped to facilitate selection of the target branch of the neurovasculature off the aortic arch as described in greater detail below. The selection catheter is available in a plurality of different tip shapes, each optimized for access into a different branch of the neurovasculature off the aortic arch. In the FIG. 1 embodiment, the disal tip section 36 is provided with a Simmons shape. Other suitable shapes include a “hockey stick shape” as well as other shapes known to those skilled in the art.

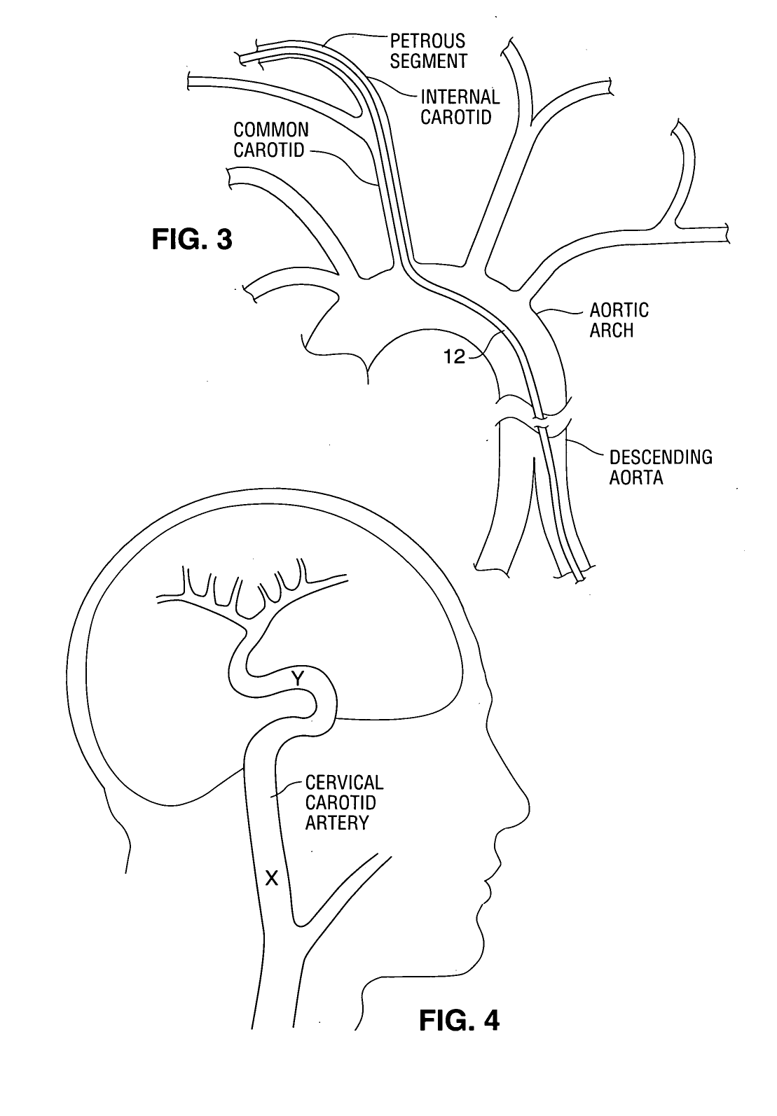

During use of the disclosed system 10, a standard sheath (not shown) is first placed in the femoral artery. The physician chooses a selection catheter 14 having a distal tip shape appropriate for selecting the target artery off the aortic arch. The delivery catheter and selection catheter are flushed with heparinized saline. A 0.018″ guidewire 16 is introduced into the selection catheter 14, and the selection catheter 14 and guidewire are introduced into the delivery catheter 12. The system is introduced into the femoral artery and advanced under fluoroscopy through the aorta into the aortic arch. The selection catheter 14 is then advanced so its distal tip shape 36 is fully deployed from the distal end of the delivery catheter 12. While pulling back and torqueing the selection catheter, the appropriate artery off the arch is selected. The delivery catheter 12 is then advanced over the selection catheter into the target artery. The selection catheter and the guidewire are then removed, and a second guidewire (e.g. a 0.038″ guidewire) is introduced into the delivery catheter. Although the first guidewire could remain in use rather than being replaced with the second guidewire, a larger diameter guidewire is preferable at this stage of the procedure (following removal of the selection catheter) because it reduces the gap between the guidewire outer diameter and the delivery catheter inner diameter and thus facilitates smoother movement of the catheter/guidewire through the tortuous vascular anatomy. The second guidewire and delivery catheter are then advanced under fluoroscopy toward the neurovascular anatomy. If the anterior circulation is chosen as shown in FIG. 3, the delivery catheter is advanced into the Petrous or Cavernous carotid artery over the second guidewire.

Interventional devices (e.g. microcatheters, stents, PTA balloon catheters, and coils) are then introduced through the delivery catheter. The advantage of the disclosed embodiment is that its soft, flexible hydrophilically coated distal end allows atraumatic advancement into the distal segments of the neurovascular anatomy. In contrast, as shown in FIG. 4, prior art delivery catheters are unable to be atraumatically advanced beyond the cervical carotid artery (position X), whereas the disclosed embodiment can pass beyond the cervical segment of the internal carotid up to the Petrous segment (position Y) or further to the Cavernous segment. When interventional devices are advanced into tortuous anatomy and encounter resistance the backward transmission of force is absorbed by the delivery catheter. The absorption of the backward force is facilitated by the distal anchored position of the delivery catheter within the angio-architecture of the neurovascular anatomy at the base of skull. In addition, the support provided by the stiff sheath-like proximal portion of the delivery catheter helps to augment the forward transmission of force. An additional advantage of the more distal placement is that it allows the physician to see the distal end of the delivery catheter and observe any back-out that may occur, and correct the situation before the delivery catheter has prolapsed into the aorta.

It should be recognized that a number of variations of the above-identified embodiments will be obvious to one of ordinary skill in the art in view of the foregoing description. Accordingly, the invention is not to be limited by those specific embodiments, methods, materials, dimensions, etc shown and described herein. Rather, the scope of the invention is to be defined by the following claims and their equivalents.

Any and all patents and patent applications referred to herein, including for purposes of priority, are incorporated herein by reference.

What is claimed is:

1. A method of accessing the intracranial vasculature, comprising the steps of:
inserting a delivery catheter percutaneously into the arterial system, and advancing a distal end of the delivery catheter to the cranial vasculature distal to the cervical segment of the internal carotid artery.

2. The method of claim 1, wherein the method includes advancing the distal end into the internal carotid artery.

3. The method of claim 2, wherein the method includes advancing the distal end at least to the Petrous segment of the internal carotid artery.

4. The method of claim 2, wherein the method includes advancing the distal end at least to the Cavernous segment of the internal carotid artery.

5. The method of claim 1, wherein advancing the distal end distal to the cervical segment of the carotid artery includes:
advancing the distal end of the delivery catheter to the aortic arch;
passing a selection catheter through the delivery catheter, the selection catheter having a shaped distal portion;
with the distal end of the delivery catheter in the aortic arch, advancing the shaped distal portion of the selection catheter from the distal end of the delivery catheter, causing the shaped distal portion to advance into a selected arterial branch distal to the aortic arch; and
advancing the delivery catheter over the selection catheter into the selected arterial branch.

6. An intracranial access system, comprising:
a delivery catheter having a delivery catheter lumen, a proximal end and a distal end, the delivery catheter including a plurality of sections, each section having greater flexibility than the section proximally adjacent to it; and
a selection catheter having selection catheter lumen and a pre-shaped distal portion, the selection catheter insertable through the delivery catheter lumen; and
at least one guidewire insertable through the selection catheter lumen.

7. The intracranial access system according to claim 6, further including instructions for use instructing the user to use the system to gain access to intracranial vasculature according to the method of claim 1, 2, 3, 4 or 5.

8. An intracranial delivery catheter comprising:
an elongate tubular member having a delivery catheter lumen, a proximal end and a distal end, the delivery catheter including a plurality of discrete sections each having a wall thickness and a tubular polymeric portion having a durometer, wherein at least one of the wall thickness and durometer of each section is lower than that of the proximally adjacent section.

9. The delivery catheter of claim 8, wherein the elongate tubular member includes a distal end having a maximum outer diameter of approximately 0.07 in.

10. The delivery catheter of claim 8, wherein the most proximal one of the plurality of sections has a length of at least approximately 81 cm.

11. The delivery catheter of claim 8, wherein the most proximal one of the plurality of sections has a tubular polymeric portion having a durometer of at least approximately 80D.

12. The delivery catheter of claim 11, wherein a distal one of the plurality of sections has a tubular polymeric portion having a durometer of 35D or lower.

13. The delivery catheter of claim 11, wherein a distal one of the plurality of sections has a tubular polymeric portion having a durometer of 80A or lower.

14. The delivery catheter of claim 8, wherein the plurality of sections includes at least two proximal sections each having a tubular polymeric portion having a durometer of 80D or higher, each of the two proximal sections having a different wall thickness.

15. The delivery catheter of claim 14, wherein the plurality of sections further includes a distal section having a tubular polymeric portion having a durometer of 80A or lower.

16. The delivery catheter of claim 15, wherein the plurality of sections further includes at least four intermediate sections between the proximal sections and the distal sections, the intermediate sections having tubular polymeric portions having durometers in the range of 72D-40D.

17. An intracranial delivery catheter comprising:
an elongate tubular member having a delivery catheter lumen and a distal end, the delivery catheter proportioned to extend intravascularly from a femoral access point in a human patient to an intra-cranial blood vessel, the elongate tubular member including:
a proximal portion including at least two rigid proximal segments, the proximal portion proportioned to extend from a femoral access point through an aortic arch;
an intermediate portion including at least two intermediate segments, the intermediate section proportioned to extend intravascularly from an aortic arch through a cervical region of the vasculature, at least one of the intermediate segments having an outer diameter smaller than the outer diameter of the at least two rigid proximal segments; and
a distal portion including at least two distal segments proportioned to extend intravascularly from a base of a skull to an intra-cranial blood vessel, at least one of the distal segments having an outer diameter smaller than the outer diameter of the at least two intermediate segments.

18. The delivery catheter of claim 17, wherein at least one of the distal segments has a maximum outer diameter of approximately 0.07 inches.

19. The delivery catheter of claim 17, wherein at least one of the proximal segments is formed of a material having a stiffness of at least approximately 80D, and wherein at least one of the distal segments is formed of a material having a stiffness of approximately 80A or less.
